(19) United States
(12) Patent Application Publication
(10) Pub. No.: US 2017/0173138 A1
(43) Pub. Date: Jun. 22, 2017

(54) IMMUNE HEALTH FOR COMPANION ANIMALS

(71) Applicant: NESTEC SA, Vevey (CH)

(72) Inventors: Fanny Bigeard, Hamelet (FR); David Claude Hernot, Amiens (FR)

(21) Appl. No.: 15/372,880

(22) Filed: Dec. 8, 2016

Related U.S. Application Data
(60) Provisional application No. 62/270,941, filed on Dec. 22, 2015.

(51) Int. Cl. A61K 39/09; A23K 20/10; A23K 50/40

(52) U.S. Cl. CPC A61K 39/09; A23K 50/40; A23K 20/10; A61K 2039/552

(57) ABSTRACT

The invention provides methods for promoting and/or improving immune health in a companion animal. In one embodiment, a method for improving immune health in a companion animal, comprising administering a composition comprising non-replicating Lactobacillus paracasei to the companion animal, wherein the composition is administered in an amount effective for increasing immune response in the animal.

### CROSS REFERENCE TO RELATED APPLICATIONS

This application claims priority to U.S. Provisional Application No. 62/270941 filed Dec. 22, 2015, the disclosure of which is incorporated herein by this reference.

### BACKGROUND OF THE INVENTION

Field of the Invention

This invention relates generally to the immune health for companion animals. In particular, the present invention relates to methods for improving immune health by increasing immune response in a companion animal.

Description of Related Art

Companion animals gain increasing socio-economic relevance for humans. There is a high demand of pet holders to have healthy and well-being companion animals for many years. In contrast to their wild-life counterparts, pets reach prolonged life time which is accompanied by appearance of same kind of diseases that afflict people.

A known strategy to compensate dietary unbalances and to prevent food-borne diseases without the use of pharmaceutical drugs is the oral administration of probiotic microorganisms. It has been demonstrated that orally administered probiotic microorganisms are able to reduce infectivity of pathogens by competing with pathogenic bacteria for nutrients and receptors for adhesion to intestinal mucus and epithelial cells. They create an environment that is inappropriate for most pathogenic bacteria resulting in general overgrowth. Additionally, some of the probiotic microorganisms secrete bactericidal components which kill pathogenic bacteria. Further, they can reduce infectivity of pathogens by stimulation of innate immune system: promoting phagocytic activity of macrophages; increased expression of complement receptor on phagocytes; activation of immature dentritic cells and production of pro-inflammatory immune modulators.

There are also reports indicating that probiotic bacteria can reduce inflammatory disorders in the gut and even suppress the establishment of allergies and cancer. Due to their presence in the gut and their metabolic activity they certainly improve the variety of innocuous antigens which are taken up by phagocytic cells and presented to the immune system. Nevertheless, the actual immunological benefit of probiotic bacteria remains controversial. Indeed, only few probiotes have been identified which are able to promote phagocytic activities and thereby improving general stimulation of the mucosal immune system.

Probiotic microorganisms are non-invasive and non-virulent. They cannot actively cross the intestinal epithelial cell layer and cannot persist in antigen presenting cells. Their immunological potential is generally rather low and depends on their uptake rate and their subsequent processing by antigen presenting cells. This may be improved by selection of probiotic bacteria with better adhesive properties to intestinal epithelial cells. However, this might also substantially increase risk of otherwise rare infections, particularly in immune compromised individuals. Significant effects have been achieved with a combination of several probiotic organisms which have been identified empirically.

In summary one can conclude that (1) only few probiotic bacteria with significant immune stimulatory efficacy do exist and (2) it is rather difficult to isolate probiotes with improved immune stimulatory efficacy. As such, there remains a need for compositions and methods to improve immune health for a companion animal.

### SUMMARY OF THE INVENTION

It is, therefore, an object of the present invention to provide methods useful for promoting and/or improving immune health care in a companion animal.

In one embodiment, a method for improving immune health in a companion animal can comprise administering a composition comprising non-replicating Lactobacillus paracasei to the companion animal, where the composition is administered in an amount effective for increasing immune response in the animal.

### DETAILED DESCRIPTION OF THE INVENTION

### Definitions

The term “companion animal” refers to any domesticated animal, and includes, without limitation, cats, dogs, rabbits, guinea pigs, ferrets, hamsters, mice, gerbils, horses, cows, goats, sheep, donkeys, pigs, and the like. In one example, the companion animal can be a dog or cat.

The term “recommended daily requirement” or “recommended daily allowance” or “RDA” refers to the daily amount of a vitamin or other nutrient recommended by a recognized agency or standard within the art including without limitation, National Institutes of Health (NIH), Institute of Medicine of the National Academies (IOM), Dietary Reference Intake (DRI) Nutrient Report, The European Pet Food Industry Federation (FEDIAF), and The Association of American Feed control Officials (AAFCO) as of Jan. 1, 2015.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. “Healthy aging animals” are those with no known diseases that might confound the results. In studies using healthy aging animals, cohort animals are also healthy aging animals,

The term “long-term administration” means periods of repeated administration or consumption in excess of one month. Periods of longer than two, three, or four can be used for certain embodiments. Also, more extended periods that include longer than 5, 6, 7, 8, 9, or 10 months can be used. Periods in excess of 11 months or 1 year can also be used. Longer term use extending over 1, 2, 3, or more years can also be included in the invention. For certain aging animals, the animal will continue consuming on a regular basis for the remainder of its life. Sometimes this is referred to as consumption for “extended” periods.

The term “regular basis” means at least monthly dosing with the compositions or consumption of the compositions, and in some aspects, weekly dosing. More frequent dosing or consumption, such as twice, three, or seven times weekly, can be used in certain embodiments. Still other embodiments include regimens that comprise at least once daily consumption. The skilled artisan will appreciate that dosing frequency will be a function of the composition that is being consumed or administered, and some compositions may require more or less frequent administration to maintain a desired level of immune health.

The term “about” includes all values within a range of 5% of the stated number. In one embodiment, “about” includes all values within a range of 2%, and in one aspect, within 1 %.

The term “individual” when referring to an animal means an individual animal of any species or kind.

As used throughout, ranges are used herein in shorthand, so as to avoid having to set out at length and describe each and every value within the range. Any appropriate value within the range can be selected, where appropriate, as the upper value, lower value, or the terminus of the range.

As used herein, embodiments, aspects, and examples using “comprising” language or other open-ended language can be substituted with “consisting essentially of” and “consisting of” embodiments.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a dog” or “a method” includes a plurality of such “dogs” or “methods”. Reference herein, for example to “a probiotic” includes a plurality of such probiotics, whereas reference to “B vitamins” includes a single B vitamin. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The methods and compositions and other advances disclosed here are not limited to particular methodology, protocols, and compositions described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

The present inventors have discovered that certain compositions can provide an immune health benefit to a companion animal. Specifically, the inventors have discovered that certain non-replicating Lactobacillus paracasei can provide improved immune health in companion animals. Additionally, methods of using non-replicating Lactobacillus paracasei to promote and/or improve the immune health care in a companion animal is provided.

In one embodiment, a method for improving immune health in a companion animal, comprising administering a composition comprising non-replicating Lactobacillus paracasei to the companion animal, wherein the composition is administered in an amount effective for increasing immune response in the animal.

Generally, non-replicating Lactobacillus paracasei can include any strain that has been heat treated such that substantially all of the bacteria are inactive. In one embodiment, at least 90% of the bacteria can be inactive. In other aspects, at least 95% of the bacteria can be inactive, at least 99% of the bacteria can be inactive, or even, at least 99.9% can be inactive. In one embodiment, the Lactobacillus paracasei can be Lactobacillus paracasei DSM 16671 (including those commercially available from BASF (PRO-T-ACTION®) or GenMont Biotech Inc. (ADP-1)).

The non-replicating Lactobacillus paracasei can be administered as a composition to the companion animal in an amount effective for promoting, treating, or improving immune health in the companion animal as discussed herein. In one embodiment, the non-replicating Lactobacillus paracasei can be administered in an amount of 0.1 mg to 50 mg per day to the companion animal. In another embodiment, the non-replicating Lactobacillus paracasei can be administered in an amount of 1 mg to 10 mg per day to the companion animal. Such compositions include any composition that can deliver non-replicating Lactobacillus paracasei. In one embodiment, the composition can be a pet food composition. In another embodiment, the composition can be a supplement. In one aspect, the composition can be delivered via a sachet.

The present NRMs can provide an increased specific and basal immune response as measured against a comparable companion animal that has not been administered the present NRMs. In one embodiment, the variant surface antigen (VSA) production of the companion animal is increased by at least 100% within 14 weeks of a first vaccination as compared to VSA production of a comparable companion animal that has not been administered the non-replicating Lactobacillus paracasei. In other aspects, the VSA can be increased by at least 10%, 25%, 50%, or even 75%. In another embodiment, the Fecal IgA of the companion animal is increased by at least 50% within 12 weeks of a first vaccination as compared to Fecal IgA of a comparable companion animal that has not been administered the non-replicating Lactobacillus paracasei. In other aspects, the Fecal IgA can be increased by at least 5%, 10%, 25%, or even 40%.

In addition to the non-replicating Lactobacillus paracasei, the composition can further comprises a member selected from the group consisting of vitamin, antioxidant, probiotic, prebiotic, and mixtures thereof.

In other embodiments, the compositions further comprise prebiotics, probiotics, or a combination thereof. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. Prebiotics include fructooligosaccharides (FOS), xylooligosaccharides (XOS), galactooligosaccharides (GOS), and mannooligosaccharides (typically for non-human foods such as petfoods). The prebiotic, fructooligosaccharide (FOS) can be found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit animal health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophillus, Bifidobacteria, Lactococcus, and Pediococcus. Beneficial species include Enterococcus and Saccharomyces species. The amount of probiotics and prebiotics to be administered to the animal is determined by the skilled artisan based upon the type and nature of the prebiotic and probiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents can be mixed with the composition or applied to the surface of the composition, e.g., by sprinkling or spraying, or be provided in a supplement.

The compositions may further comprise substances such as minerals, vitamins, salts, nutrients, functional additives including, for example, palatants, colorants, emulsifiers, antimicrobial or other preservatives. Minerals that may be useful in such compositions include, for example, calcium, phosphorous, potassium, sodium, iron, chloride, boron, copper, zinc, magnesium, manganese, iodine, selenium and the like. Examples of additional vitamins useful herein include vitamin B and such fat soluble vitamins as D and K. Inulin, amino acids, taurine, choline, enzymes, coenzymes, and the like may be useful to include in various embodiments.

The B vitamins can be any B vitamin suitable for administration to an animal. B vitamins include vitamin B1 (thiamine), vitamin B2 (riboflavin), vitamin B3 (niacin, nicotinic acid, nicotinamide), vitamin B5 (pantothenic acid), vitamin B6 (pyridoxine, pyridoxal, pyridoxamine), vitamin B7 (biotin), vitamin B8 (myo-inositol), vitamin B9 (folic acid) vitamin B12 (cobalamin compounds including methylcobalamin, hydroxocobalamin, and cyanocobalamin), or salts, conjugates, or derivatives thereof recognized of found to have B vitamin activity. Combinations of any of the foregoing are also useful herein and are sometimes referred to herein as “mixtures” of B vitamins.

The antioxidants can be any antioxidant suitable for administration to an animal. Antioxidants are well known in the art, particularly the art of food technology and food formulation. Natural antioxidant compounds include vitamins (such as A, C and E, and derivative, conjugates, or analogs thereof), as well as plant extracts, including extracts from fruit, vegetables, herbs, seeds, and other types and/or parts of plants. Compounds such as α-lipoic acid, chlorophyll and derivatives thereof, glutathione, ubiquinols (e.g., coenzyme Q10), carotenoids (e.g., beta-carotene, alpha-carotene, beta-cryptoxanthin, gamma-carotene, lutein, astaxanthin, and zeaxanthin etc.), flavonoids, phenolic acids and polyphenols, and pycnogenol are known to be excellent antioxidants. Some examples of plant sources of antioxidants include those from fruits such as berries (cherry, blackberry, strawberry, raspberry, crowberry, blueberry, bilberry/wild blueberry, black currant), pomegranate, grape, orange, plum, pineapple, kiwi fruit, and grapefruit; those from vegetables including kale, chili pepper, red cabbage, peppers, parsley, artichoke, Brussels sprouts, spinach, lemon, ginger, garlic, and red beets; those from dry fruits like apricots, prunes, and dates; from legumes including broad beans, pinto beans, and soybeans. Also nuts and seeds such as pecans, walnuts, hazelnuts, ground nut, and sunflower seeds; cereals such as barley, millet, oats, and corn. Many natural antioxidants are also available from a wide variety of spices including cloves, cinnamon, rosemary, and oregano. Less widely known sources of antioxidants include Ginkgo biloba, and tropical plants such as uyaku, and carica papaya. Antioxidant properties of various teas and green tea, as well as fermented products such as red wine, have become of great interest in recent years and such would be suitable for use herein. Selenium is an excellent oxygen scavenger and works well, especially with vitamin or related tocopherol compounds. Synthetic dietary antioxidants include butylated hydroxyanisole (BHA) and butylated hydroxytoluene (BHT) which are commonly used in food products. Any of the foregoing, alone or in combination, are suited for use herein, as are combinations of natural and synthetic antioxidants. In one embodiment, the antioxidants comprise astaxanthin alone or in combination with other antioxidants.

Additionally, the composition comprises from about 0.1 to 40 times the recommended daily requirement (RDA) of minerals and vitamins and from about 0.0001% to about 25% of antioxidants. In various embodiments, the minerals and vitamins comprise from about 0.1 to 20 times the RDA, and in one aspect, from about 0.1 to 10 times the RDA, and antioxidants comprise from about 0.0001% to about 15%, in one aspect, from about 0.001% to about 5%, and in one specific aspect, from about 0.001% to about 2%.

In one embodiment, the compositions can be pet food compositions. Such compositions include foods intended to supply the necessary dietary requirements for an animal, animal treats (e.g., biscuits), or dietary supplements. The compositions may be a dry composition (e.g., kibble), semi-moist composition, wet composition, or any mixture thereof. In another embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the non-replicating Lactobacillus paracasei, probiotics, prebiotics, minerals, vitamins and/or antioxidants. This permits the supplement to be administered to the animal in small amounts, or in the alternative, can be diluted before administration to an animal. The dietary supplement may require admixing, or can be admixed with water or other diluent prior to administration to the animal.

In one embodiment, the compositions can be refrigerated or frozen compositions. In another embodiment, the non-replicating Lactobacillus paracasei can be pre-blended with the other components to provide the beneficial amounts needed. In yet other embodiments, the non-replicating Lactobacillus paracasei can be used to coat a food, snack, pet food composition, or pet treat. In one embodiment, the non-replicating Lactobacillus paracasei can be added to the composition just prior to offering it to the animal, e.g., using a sprinkled powder or a mix. Such compositions further comprise probiotics, prebiotics, minerals, vitamins and/or antioxidants.

The compositions can optionally comprise one or more supplementary substances that promote or sustain general health. Preferred substances may be associated with improved immune health or may be substances that promote immune health.

In various embodiments, pet food or pet treat compositions can comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the composition ranges from less than 1% to about 15%, and in one aspect, from about 5% to about 10%.

The moisture content can vary depending on the nature of the composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for companion animals.

The compositions may also comprise one or more fiber sources. The term “fiber” includes all sources of “bulk” in the food whether digestible or indigestible, soluble or insoluble, fermentable or nonfermentable. Fibers can be from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, fructooligosaccharide, pectin, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present invention to the immune system of an animal.

The compositions and dietary supplements may be specially formulated for the intended recipients or consumers, such as for adult animals or for older or young animals. For example, a composition adapted for puppies or kittens or adapted for active, pregnant, lactating, or aging animals can be prepared. In general, specialized compositions will comprise energy and nutritional requirements appropriate for animals at different stages of development or age.

Certain aspects of the invention can be used in combination with a complete and balanced food. According to certain embodiments provided herein, the compositions comprising the non-replicating Lactobacillus paracasei, probiotics, prebiotics, minerals, vitamins, and/or antioxidants, can be used with a high-quality commercial food. As used herein, “high-quality commercial food” refers to a diet manufactured to produce the digestibility of the key nutrients of 80% or more, as set forth in, for example, the recommendations of the National Research Council above for dogs, or in the guidelines set forth by the Association of American Feed Control Officials. Similar high nutrient standards would be used for other animals.

In one embodiment, the food compositions comprise any of a variety of ingredients or combinations thereof selected for their contributions to the overall composition. Thus a skilled food technologist may choose from among natural (e.g., plant or plant-derived, animal or animal-derived, and microbial or microbially-derived), and synthetic ingredients or components. In particular embodiments, the ingredients may include any of the cereal grains and/or fractions or components thereof, meat and meat by-products, fish, shellfish, or other seafood, other animal products or by-products, eggs from any source, vitamins, minerals, salts, sweeteners, fiber, flavoring or other palatants, coloring, and functional ingredients such as emulsifiers, stabilizers, softeners, functional coatings, and the like. Cereals useful in the invention include all plants recognized as “cereal” crops, whether currently used in commercial agriculture or merely known practically or botanically as being a “cereal”. For example, “cereals” includes corn, wheat, rice, barley, sorghum, millet, oats, rye, triticale, buckwheat, fonio, and quinoa. The skilled artisan will appreciate that in a given food composition, it is not uncommon to use one or more such cereal products. Meats useful in the invention include products from any animal, including muscle tissue such as chicken or other poultry, lamb, sheep, veal, beef, or pork. Other animal products and by-products useful in the invention include dairy products or by-products derived from the milk of any species. Other important components or ingredients include fats and the skilled artisan will appreciate that many sources of vegetable, animal, or microbial fats are available for formulating food compositions. In one embodiment, the source of fat is a plant fat such as corn, soy, or canola oil, including those that are readily available. In another embodiment, an animal fat, such as tallow, is useful for providing calories from fat, as well as enticing flavor to meat-eating animals. Of course, combinations of any of the foregoing ingredients, such as fats, are known in the art and useful for optimizing the food compositions based on functional properties as well as price and availability.

The skilled artisan will also appreciate that in formulating the food compositions of the invention, the formulation may vary slightly, so as to allow consideration by the formulator of the price and/or availability of certain ingredients in the compositions, as well as the batch-to-batch variation in the analysis of certain ingredients. Thus a given food composition or formulation may vary slightly from batch to batch, plant to plant, or even season to season depending on such factors. Notwithstanding such variation in specific ingredients selected for manufacturing a particular batch of a food composition, the overall composition (for example, analysis of protein, carbohydrate, fat, fiber, or other component) may be held constant or at least substantially constant, for example, in accordance with a label claim, such as a claim or guarantee of a minimum or maximum percent of a particular component.

In one embodiment, the food compositions comprise a macronutrient composition suitable for the type of food being designed. In one embodiment, the food composition can have about 20 to 32% protein, about 30 to 50% carbohydrate, about 5% to 20% fat, and about 15% to 25% moisture. In another embodiment, the food composition can be a pet food composition such as a premium or super-premium pet food composition. In one embodiment, the pet food can be formulated for canines and has a protein content of about 20-30%, in one aspect, about 24-28%, and in one specific aspect, about 25-27%. In one embodiment, the protein content of a dog food composition can be about 26% by weight. In another embodiment, the formulation can be for felines and can have a protein content of about 35-45%, in one aspect, about 37-42%, and in one specific aspect, about 39-41%. In one embodiment, the protein content of a cat food composition can be about 40%. In another embodiment, the composition can be a food product comprising non-replicating Lactobacillus paracasei, vitamins, and antioxidants, and further comprising about 15% to about 50% protein, about 5% to about 40% fat, about 5% to about 10% ash content, and having a moisture content of about 5% to about 20%.

In one embodiment, the food composition can be a wet food, such as a canned food, frozen food, or fresh food product. In one embodiment, the food composition can be shelf stable. In another, it can be refrigerated. In other embodiments, the food composition can be an intermediate moisture product or a dry food product as described above.

In another aspect, the invention provides pharmaceutical compositions comprising a composition of the present invention and one or more pharmaceutically-acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals.

Administration in accordance with the methods can be on an as-needed or as-desired basis of varying or regular frequency. A goal of regular ingestion is to provide the animal with a regular and consistent dose of the composition. Such regular and consistent dosing will tend to provide for improved immune health. Thus, administration on a regular basis can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal, e.g., orally, or otherwise. The compositions can alternatively be contacted with, or admixed with, daily feed or food, including a fluid, such as drinking water, or an intravenous connection for an animal that is receiving such treatment. When utilized as a daily feed or food, administration will be well known to those of ordinary skill.

Administration can also be carried out as part of a dietary regimen for the animal. For example, a dietary regimen may comprise causing the regular ingestion by the animal of a composition described herein in an amount effective to promote or improve the immune health in the animal.

According to the methods of the invention, administration of the compositions, including administration as part of a dietary regimen, can span a period of time ranging from parturition through the adult life of the animal. In various embodiments, the companion animal can be a dog or cat. In certain embodiments, the animal can be a young or growing animal. In other embodiments, the animal can be an aging animal. An animal that has reached about 35% of its projected lifespan can be included. In some embodiments, administration begins, for example, on a regular or extended regular basis, when the animal has reached more than about 30%, 40%, or 50% of its projected or anticipated lifespan. In some embodiments, the animal can have attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

In a further aspect, the invention provides kits suitable for administering a composition comprising non-replicating Lactobacillus paracasei to a companion animal. The kits comprise in separate containers in a single package or in separate containers in a virtual package, as appropriate for the kit component, (a) non-replicating Lactobacillus paracasei, (b) a mineral, (c) a vitamin, and/or (d) an antioxidant; and (1) other ingredients suitable for consumption by an animal, (2) a prebiotic, (3) a probiotic, (4) instructions for how to combine or prepare the non-replicating Lactobacillus paracasei and any other ingredients provided in the kit for administration to an animal; (5) instructions for how to use the combined kit components, prepared kit components, or other kit components for the benefit of an animal; and/or (6) a device for administering the combined or prepared kit components to an animal. The components are each provided in separate containers in a single package or in mixtures of various components in different packages. The kits may comprise the ingredients in various combinations. For example, the kit could comprise a mixture of non-replicating Lactobacillus paracasei, vitamins and antioxidants in one container and one or more other ingredients in one or more other containers. Other such combinations can be produced by the skilled artisan based upon the characteristics of the ingredients and their physical and chemical properties and compatibilities.

In another aspect, the invention provides a means for communicating information about or instructions for one or more of (1) using compositions of the present invention for promoting or improving immune health; (2) admixing the non-replicating Lactobacillus paracasei, vitamins, antioxidants, minerals, or other components of the invention to produce a composition suitable for promoting or improving immune health; (3) using the kits of the present invention for promoting or improving immune health; and (4) administering the compositions to an animal. The means comprises one or more of a physical or electronic document, digital storage media, optical storage media, audio presentation, audiovisual display, or visual display containing the information or instructions. In one embodiment, the means can be selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising non-replicating Lactobacillus paracasei, and one or more other ingredients suitable for consumption by an animal, e.g., protein, fat, carbohydrate, fiber, etc. The methods comprise admixing one or more ingredients suitable for consumption by an animal with non-replicating Lactobacillus paracasei, and possibly other ingredients such as vitamins and/or antioxidants. Alternatively, the methods comprise applying non-replicating Lactobacillus paracasei, and other ingredients if desired, separately or in any combination onto the food composition, e.g., as a coating or topping. The non-replicating Lactobacillus paracasei can be added at any time during the manufacture and/or processing of the food composition. This includes, for example, admixing the non-replicating Lactobacillus paracasei as part of the core formulation of the “body” of the food composition or applying them as a coating, i.e., primarily to the surface of the food composition after its manufacture. The compositions can be made according to any method suitable in the art.

In another aspect, the present invention provides a package comprising a composition of the present invention and a label affixed to the package containing a word or words, picture, design, acronym, slogan, phrase, or other device, or combination thereof, that indicates that the contents of the package contains a composition suitable for promoting and/or improving immune health in an animal. Typically, such device comprises the words “improves immune health”, or an equivalent expression printed on the package. Any package or packaging material suitable for containing the composition can be useful in the invention, e.g., a bag, box, bottle, can, pouch, and the like manufactured from paper, plastic, foil, metal, and the like. In a one embodiment, the package can contain a food composition adapted for a particular animal such as a canine or feline, as appropriate for the label, in one aspect, a companion animal food composition.

In the invention, the animal can be a juvenile, adult, senior, or geriatric animal. In one embodiment, the animal can be an aging animal. Generally, animals can be senior in the last half of their expected lifespan and geriatric in the last fourth of their expected lifespan. Lifespan definitions vary for various animals and are known to skilled artisans. For a dog or cat, the animal can be considered to be juvenile until 1 year of age.

### EXAMPLES

The invention can be further illustrated by the following example, although it will be understood that this example is included merely for purposes of illustration and is not intended to limit the scope of the invention unless otherwise specifically indicated.

### Example 1

### Immune Study for Felines for Non-replicating Microorganisms (NRM)

An immune study for felines was performed using 15 healthy adult cats. Effects of NRMs: a mixture composed of Lactobacillus fermentum and Lactobacillus delbrueckii (NCC3066 from Konolfmgen) and Lactobacillus paracasei DSM 16671 (PRO-T-ACTION® pet grade from BASF) on immune function and microflora were studied vs a control. A standard diet was used as a control that was supplemented with either the mixture of Lactobacillus fermentum and Lactobacillus delbrueckii (17 mg) or Lactobacillus paracasei DSM 16671 (5 mg) for daily delivery. The results for Fecal IgA (ug/mL)—Basal Immune Response is provided in Table 1 and the results for VSA (IU/mL)—Specific Immune Response is provided in Table 2.

TABLE 1

| Time after first | Control | NCC3066 | PRO-T-ACTION ® |
|---|---|---|---|
| vaccination | Fecal IgA | Fecal IgA | Fecal IgA |
| (weeks) | (ug/mL) | (ug/mL) | (ug/mL) |
| 0 | 502 | 545 | 631 |
| 12 | 461 | 405 | 711 |

TABLE 2

| Time after first | Control | NCC3066 | PRO-T-ACTION ® |
|---|---|---|---|
| vaccination | VSA | VSA | Fecal VSA |
| (weeks) | (IU/mL) | (IU/mL) | (IU/mL) |
| 0 | 0 | 0 | 0 |
| 14 | 8.8 | 13.0 | 23.9 |
| 16 | 26.5 | 18.0 | 29.0 |

Notably, as shown in Table 1, at T12, PRO-T-ACTION® showed significantly higher IgA level compared to both group control (p<0.10) and NCC3066 (p<0.05). Additionally, as shown in Table 2, after first vaccination, the VSA production in the PRO-T-ACTION® group was higher than the Control and NCC3066 groups. This shows a faster/stronger immune response.

In the specification, there have been disclosed typical embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for improving immune health in a companion animal, comprising administering a composition comprising non-replicating Lactobacillus paracasei to the companion animal, wherein the composition is administered in an amount effective for increasing immune response in the animal.

2. The method of claim 1, wherein the Lactobacillus paracasei is Lactobacillus paracasei DSM 16671.

3. The method of claim 1, wherein the non-replicating Lactobacillus paracasei is administered in an amount of 0.1 mg to 50 mg per day to the companion animal.

4. The method of claim 1, wherein the non-replicating Lactobacillus paracasei is administered in an amount of 1 mg to 10 mg per day to the companion animal.

5. The method of claim 1, wherein the variant surface antigen (VSA) production of the companion animal is increased by at least 100% within 14 weeks of a first vaccination as compared to VSA production of a comparable companion animal that has not been administered the non-replicating Lactobacillus paracasei.

6. The method of claim 1, wherein the Fecal IgA of the companion animal is increased by at least 50% within 12 weeks of a first vaccination as compared to Fecal IgA of a comparable companion animal that has not been administered the non-replicating Lactobacillus paracasei.

7. The method of claim 1, wherein the composition is a pet food composition.

8. The method of claim 1, wherein the composition is a supplement.

9. The method of claim 1, wherein the composition further comprises a member selected from the group consisting of vitamin, antioxidant, probiotic, prebiotic, and mixtures thereof.

10. The method of claim 1, wherein the composition further comprises a prebiotic or probiotic.

11. The method of claim 10, wherein the prebiotic is selected form the group consisting of fructooligosaccharides (FOS), xylooligosaccharides (XOS), galactooligosaccharides (GOS), mannooligosaccharides, and mixtures thereof.

12. The method of claim 10, wherein the probiotic is selected from the group consisting of Lactobacilllus acidophillus, Bifidobacteria, Lactococcus, Pediococcus, and mixtures thereof.

13. The method of claim 1, wherein the animal is a canine.

14. The method of claim 1, wherein the animal is a feline.

15. The method of claim 1, wherein the composition is administered on a regular basis.
